(19) United States
(12) Patent Application Publication
(10) Pub. No.: US 2014/0227768 A1
(43) Pub. Date: Aug. 14, 2014

(54) NOVEL APPARATUS AND METHOD TO PROPAGATE BIOLOGICAL MATERIAL

(71) Applicant: MOHAMMAD AHMED KHIYAMI, RIYADH (SA)

(72) Inventor: MOHAMMAD AHMED KHIYAMI, RIYADH (SA)

(73) Assignee: King Abdulaziz City for Science and Technology (KACST), Riyadh (SA)

(21) Appl. No.: 14/093,657

(22) Filed: Dec. 2, 2013

Related U.S. Application Data
(63) Continuation-in-part of application No. 13/762,635, filed on Feb. 8, 2013, now Pat. No. 8,691,521.

(51) Int. Cl. C12M 1/26

(52) U.S. Cl. CPC C12M 33/18
USPC 435/252.1; 435/309.1

(57) ABSTRACT

The present application discloses a novel microbiological apparatus and methods of using the same. The apparatus is a rolling pen comprising of a socket and a ball with its unique rolling head to transfer a biological material from a first location to a second location in an aseptic manner. Further, the rolling pen apparatus can be used with a pen holder device or with a holder hand device to transfer the biological material a first location to a second location and further to develop a microbiological colony. The invention also discloses the use of rolling pen apparatus attached to a holder hand device in propagating a single biological colony such as bacterial colony or cells.

### CROSS REFERENCE TO RELATED APPLICATION

The instant application is a continuation in part application and claims priority to pending U.S. patent application Ser. No. 13/762,635, filed on 8 Feb. 2013. The disclosure is hereby incorporated by this reference in its entirety for all of its teachings.

### FIELD OF TECHNOLOGY

This disclosure generally relates to an apparatus and method for transferring and propagating biological material. More specifically, the present disclosure relates to an apparatus and method for transferring and propagating biological materials in aseptic conditions.

### BACKGROUND

Microbiological sample culturing and sub-culturing experiments needs highly trained and skilled professionals, with an ability to operate the tools while maintaining aseptic conditions. The transfer of a microbiological colony may be a multi-step or a single step process depending on the tools and the experimental protocol. Often the lack of good aseptic practices is a problem for such microbiological experiments. Routinely used tools to transfer microbiological colony include inoculating needles and loops (plastic and/or metal), pipette tip or toothpick. The loops as mentioned may scratch the media surface and result in an uneven biological sample growth and will eventually affect the results outcome.

Further, automated equipment's are also being used at an industrial level for streaking purpose, in order to achieve better sterilization and consistency. This helps in reliable and faster work. However, the automated method is convenient in large scale industrial settings but proves costly for a small business and routine laboratory work. There is still a need for more efficient tools for small scale applications.

### SUMMARY

The present invention discloses an apparatus and a method to transfer and propagate a biological material from a first location to a second location in an easy and convenient way under aseptic conditions. The instant application is an improvement over the prior related application. In some embodiments, the disclosure presents an apparatus to transfer a microbiological colony such as bacterial colony from a first location to a second location in an easy and convenient way under aseptic conditions. In one embodiment, the disclosure presents a method of using the disclosed apparatus for spreading/streaking the microbiological colony for propagation to almost a single microbiological colony. In another embodiment, the disclosure presents a method of using the disclosed apparatus for spreading/streaking the microbiological colony for growing microbiological cultures.

In one embodiment, the first location is a first place where the biological material such as a microbiological colony is present. In another embodiment, the second location is a second place where the biological material such as a microbiological colony or cells is transferred for growth. The first location may be a water source, soil, food, cultures on petri plate, culture tube etc. The second location where the microbiological colony is transferred for growth may be a petri plate, a culture tube or other surface suitable for growth. In most embodiments, a biological material is a biological colony such as bacteria or cells.

The invention also discloses a method of using the apparatus by different devices for easy handling and use of the disclosed apparatus.

In most embodiments, the apparatus as disclosed is a rolling pen apparatus. The rolling pen apparatus as disclosed comprise of a socket and a ball. The socket is a hollow body from inside with a proximal end and a distal end. The proximal end has a diameter less than the distal end of the socket. In one embodiment, the socket is in a shape of a trapezoid or a cup which is hollow from inside. In another embodiment, the socket may have another geometrical shape.

In one embodiment, the ball may be inserted into the proximal end of the socket whereas in another embodiment, the ball may be inserted into the distal end of the socket. In most embodiments, the ball is inserted into the socket in a single rolling pen apparatus. In one embodiment, the socket may encircle a three-fourth portion through its proximal end or a one half portion through its proximal end. At any time a portion of the ball is inserted into the socket and a portion of the ball is present outside of the socket. In some other embodiments, the diameter of the socket will vary with the diameter of the ball. The diameter of the proximal end of the socket will always be more than the diameter of the ball.

In one embodiment, the ball as disclosed further comprise of a head. In another embodiment, the ball as disclosed further comprise of a rolling head. The rolling head of the ball helps in moving the ball in a backward direction, a forward direction or in other desired direction during its use. In one embodiment, the rolling head of the ball helps in even spreading/streaking/propagating of the biological or microbiological colony onto a solid media for growth. In another embodiment, the rolling head of the ball helps in the transfer of the microbiological colony from the first location to the second location. In most embodiment, the rolling head of the ball helps in the transfer of the bacterial/cell culture from the first location to the second location. The rolling pen apparatus with a socket and a ball is a single rolling pen apparatus and may be referred same throughout the application. The ball may rotate for 360 degrees and is easy to sterilize.

In one embodiment, the ball with its rolling head helps in forming a single microbiological colony by a point spreading/streaking of a microbiological colony. In another embodiment, the ball with its rolling head helps in forming a single bacterial colony by a point spreading/streaking of a bacterial culture. In most embodiments, the ball with its rolling head helps in spreading/streaking a biological and/or a microbiological colony evenly over a surface or second location where it is to be transferred. The rolling head of the ball sticks to a biological material on its tip and then it may be transferred to a second location or spread evenly over a solid media or surface for growth. Thus, one of the advantages of the present disclosure is the ability to develop a single colony of a biological/microbiological/cell colony. An apparatus of this kind has not been described in such a simple and easy way before.

In one embodiment, the rolling pen apparatus may be pre-sterilized and stored as a single pack or may be supplied as a large sterilized package. In another embodiment, the rolling pen apparatus may be sterilized before running the experiment, in between the experiment and after the experiment depending on the experimental needs and the rolling pan apparatus used.

In one embodiment, a rolling pen apparatus is disclosed comprising of a socket and a ball to transfer a microbiological colony from a first location to a second location. In another embodiment, a rolling pen apparatus is disclosed comprising of a socket and a ball to spread a microbiological colony for microbiological culture growth. In most embodiments, a rolling pen apparatus is disclosed comprising of a socket and a ball to develop single microbiological and/or bacterial colony.

In most embodiments, the present invention discloses a pen holder device to hold the rolling pen apparatus for its functioning. In one embodiment, the rolling pen apparatus may be loaded into and used with a pen holder device to transfer the biological material from a first location to a second location. In another embodiment, the rolling pen apparatus may be loaded into and used with a pen holder device to transfer a microbiological colony such as a bacterial culture from a first location to a second location. The rolling pen apparatus may be loaded as a single unit at any time of the experiment or a number of units one over another as a stack in the pen holder device for its use.

In one embodiment, the pen holder device comprises of a double-walled barrel shaped chamber called as a pen chamber. In another embodiment, the barrel shaped chamber or a pen chamber has an inner wall and an outer wall. The pen holder device further comprises of an ON/OFF switch. In one embodiment, the ON/OFF switch may be present on the outer wall of the pen holder device. In another embodiment, the ON/OFF switch may be present at the end of the outer wall of the pen holder device. The pen holder device further comprise of a proximal end and a distal end. The distal end of the pen holder device comprise of an open side which may be opened and closed to load/unload the rolling pen apparatus into the pen holder device. The distal end of the pen holder device also comprise of a eject button. The eject button can be pushed to lower the rolling pen apparatus into the pen chamber and also to eject the rolling pen apparatus from the pen chamber once the experiment is complete or once the use of the rolling pen apparatus is complete.

The pen holder device further comprise of a UV light source which may be present on the outer wall of the pen chamber or in the inner wall of the pen chamber. The UV light source may also be present at the proximal end or at the distal end of the pen holder device. The UV light source helps in sterilizing the pen chamber before and after the experiment/use along with sterilizing/re-sterilizing the rolling pen apparatus. The ON/OFF switch helps to control the function and time of exposure of the UV light source.

In most embodiments, a pen holder device is disclosed comprising: a pen chamber with an inner wall and an outer wall; a UV light source located on the outer wall; a proximal end; and a distal end wherein the pen holder holds the rolling pen apparatus for its use.

In most embodiments, the present invention discloses a holder hand device which may be used to hold a rolling pen apparatus for its functionality. In one embodiment, the rolling pen apparatus may be attached to a holder hand for its use. In another embodiment, the rolling pen apparatus may be fixed into a holder hand for its use. The holder hand as disclosed comprise of a holder and a long-extended hand. In one embodiment, the holder is cylindrical in shape whereas in another embodiment, the holder is in the shape as of the rolling pen apparatus. The holder as disclosed may be hollow from inside. In one embodiment, the diameter of the holder is larger than the diameter of the ball of the rolling pen apparatus so that the ball of the rolling pen apparatus can fit nicely into the holder of the holder hand device. In some embodiments, the diameter of the holder is larger than the diameter of the outer wall of the socket so that the rolling ball apparatus may be inserted or attached inside of the holder of the holder hand device. Further, the hand of the holder hand device is a long, extended body.

In some embodiments, the holder hand device has a hinge attached into it. The hinge helps in the attachment of the holder and the long extended hand. Further, the hinge also provides flexibility to the holder hand device. In one embodiment, the flexibility of the hinge helps in picking up and transferring a biological material whereas in another embodiment, the flexibility of the hinge aid in spreading a biological material onto a solid media surface. In one embodiment, the long extended hand may be in a horizontal position or a vertical position with respect to the holder. In another embodiment, the long extended hand may be in a position ranging between 0 to 90 degrees with respect to the holder. In some embodiments, the hand may also be fixed at a defined angle for the user's comfort. A user may hold the holder hand device via the long extended hand for use and thus move the holder hand device with a rolling pen apparatus in a desired direction during the microbiological experiments. In one embodiment, the rolling pen apparatus is attached to the holder hand device as a single unit whereas in another embodiment, the rolling pen apparatus and the holder hand device are two separate units which may be combined at the time of the experimentation to make it a single unit.

Further, the invention also discloses a use of the rolling pen apparatus. In one embodiment, a use of the rolling pen apparatus in the transfer of biological material from a first location to a second location is disclosed. In another embodiment, a use of the rolling pen apparatus for spreading the biological material for culture growth is disclosed. In one embodiment, the rolling pen apparatus may be loaded into a pen holder device for its use. In another embodiment, the rolling pen apparatus may be used as an attachment with a holder hand device for its use. In some embodiments, the rolling pen apparatus is loaded as a single unit or as a number of units into the pen holder device for use which are then pushed one by one as they are used. In most embodiments, a method of using the rolling pen apparatus with a pen holder device or with a holder hand device is disclosed.

In one embodiment, a method of using a rolling pen apparatus with a pen holder device to transfer a biological material or a microbiological colony is disclosed. As disclosed the method comprise of opening a proximal end of a pen holder device; disinfecting a pen chamber; loading the rolling pen apparatus into the pen chamber for use; turning ON the UV light to sterilize or re-sterilize the rolling pen apparatus, the pen chamber and the distal end of the pen holder device; turning OFF the UV light source; pushing the eject button to lower the rolling pen apparatus unit into the pen chamber; placing the rolling pen apparatus near a biological material to be transferred from a first location; picking up the biological material; transferring the biological material onto a second location; pushing the eject button again to discharge the used rolling pen apparatus.

In some embodiments, a method of using a rolling pen apparatus with a holder hand device to develop microbiological cultures is disclosed. The method comprises: attaching an un-sterilized or a pre-sterilized unit of the rolling pen apparatus with a holder hand device; pointing a ball of the rolling pen apparatus towards microbiological culture; picking up the microbiological culture with the rolling pen apparatus from a first petri plate; spreading evenly the microbiological culture onto a solid media surface of a second petri plate; growing the microbiological cultures.

In some embodiment, a method of using a rolling pen apparatus with a holder hand device to develop single bacterial colony is disclosed. As disclosed, the method comprises: attaching an un-sterilized or a pre-sterilized unit of the rolling pen apparatus with a holder hand device; pointing a ball of the rolling pen apparatus towards a bacterial colony; picking up the bacterial colony with the ball of the rolling pen apparatus from a first petri plate; placing dots of the bacterial colony into a second petri plate; growing of a single bacterial colony.

Thus, the rolling pen apparatus as disclosed may be used with a pen holder device or with a holder hand device as per a user's choice, convenience, experimentation requirements, microbiological sample to be used and experimentation conditions among other deciding factors.

Other features will be apparent from the accompanying figures and from the detailed description that follows.

### BRIEF DESCRIPTION OF THE DRAWINGS

Example embodiments are illustrated by way of example and no limitation in the accompanying figures indicate similar elements and in which:

FIG. 1 shows a rolling pen apparatus.

FIG. 2 shows a pen holder device to use a rolling pen apparatus.

FIG. 3 shows a holder hand device to use a rolling pen apparatus.

FIG. 4 shows a holder hand device with a rolling pen apparatus attached to it.

FIG. 5 shows a side view of a holder hand device.

FIG. 6 (a) shown a holder hand device with a rolling pen apparatus attached to a bacterial culture in a petri plate.

FIG. 6 (b) shows the spreading of the bacterial culture on a solid media in a petri plate.

FIG. 6 (c) shown the development of single bacterial colony onto the solid media in a petri plate.

FIG. 7 shows a flow-chart representation of different methodologies of using a rolling pen apparatus

FIG. 8 shows a flow-chart of a method of using a rolling pen apparatus with a pen holder device.

FIG. 9 shows a flow-chart of a method of using a rolling pen apparatus with a holder hand device.

Other features of the present embodiments will be apparent from the detailed description that follows.

### DETAILED DESCRIPTION

Several embodiments of the presently disclosed application describe an apparatus for the growth of biological material such as transfer of microbiological colony from a first location to a second location and its method of use. Further, embodiments of the presently disclosed application also describe the use of the apparatus for a single bacterial/cell colony growth.

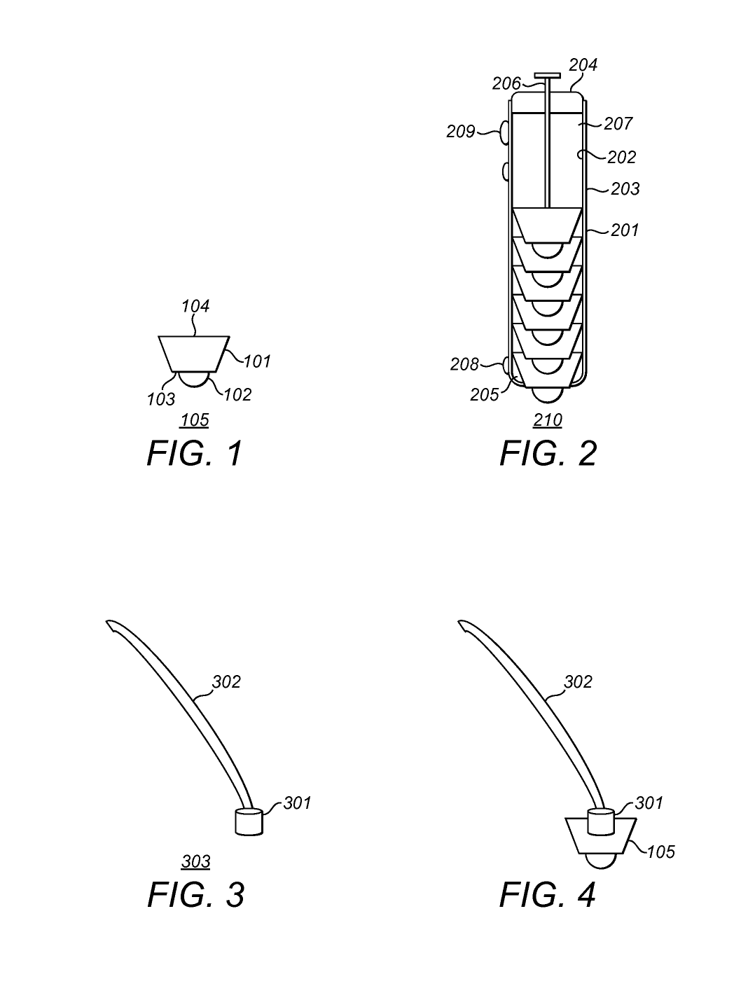

FIG. 1 shows a rolling pen apparatus. The rolling pen apparatus comprise of a socket 101 and a ball 102 inserted within the socket. The socket 101 as shown comprises of a proximal end 103 and a distal end 104. The diameter of the proximal end 103 is less than the diameter of the distal end 104. The socket is in a shape of a cup or a trapezoid which is hollow from inside and differs in their diameter. The socket may be in any other geometrical shape with the above mentioned specifications. The ball 102 is inserted into the proximal end 103 of the socket 101. The proximal end 103 of the socket 101 houses the ball such that it may cover three fourth portion of the ball 102. Further, the proximal end 103 of the socket 101 may cover half or two third portion of the ball depending on the diameter of the ball and the diameter of the proximal end 103 of the socket 101. The socket also comprise of an inner wall and an outer wall.

The ball 102 as disclosed comprise of a rolling head. Thus, the ball can be rolled in a backward direction, a forward direction or any other desired direction depending on the function being performed by the rolling pen apparatus. The rolling head of the ball gives mobility to the rolling pen apparatus and makes it easy to move the rolling pen apparatus in a desired direction. The rolling pen apparatus function in picking biological material such a microbiological colony from a first location and helps it to transfer to a second location. Further, the rolling pen apparatus also helps in the spreading of the microbiological colony onto a desired media for a microbiological culture growth. The rolling head of the rolling pen apparatus helps in picking up the microbiological colony such as bacteria from one location and to evenly spread it over a solid media surface for the bacterial culture growth. The tip of the rolling head in the rolling pen apparatus picks up the biological or microbiological colony such as bacteria. The rolling head of the rolling pen apparatus further aid in point spreading by placing a dot of the bacterial culture over the solid media for a single bacterial colony growth. Moving the ball within the socket onto the solid media helps in minimizing the load of the bacteria and thus results in getting a single colony. One of the advantages of the rolling head in the rolling pen apparatus is in developing a single bacterial colony.

The first location is a first place where the biological material such as a microbiological colony is present. The second location is a second place where the biological material such as a microbiological colony is transferred for growth. The first location may be a water source, soil, food, cultures on petri plate, culture tube etc. The second location where the microbiological colony is transferred for growth may be a petri plate, a culture tube or other surface suitable for growth.

The rolling pen apparatus may be manufactured both as a pre-sterilized units or may be an un-sterilized unit. The rolling pen apparatus may be made of a plastic material, a metal or any other suitable material. Preferably the rolling pen apparatus may be made of a plastic material and supplied as pre-sterilized.

Rolling pen apparatus may be used via a pen holder device as shown in FIG. 2 for its functions. The pen holder device comprise of a long barrel shape pen chamber 201. The pen chamber 201 has an inner wall 202 and an outer wall 203. The chamber is hollow from inside. Further, the pen chamber has a distal end 204 and a proximal end 205. The distal end further comprise of a eject button 206 which may be operated by a user's thumb with ease to lower the rolling pen apparatus during use and/or eject the rolling pen apparatus from the pen chamber after its use. The distal end comprise of an open side 207 which opens to load/unload the rolling pen apparatus into the pen chamber 201. The inner wall of a pen chamber may be made up of a UV protected material such as ceramic, aluminum and synthetic material. The outer wall of pen chamber is further equipped with a UV light source 208. There is an ON and OFF switch 209 to control the UV light source. The rolling pen apparatus is lowered from the open proximal end with its ball 102 head pointing towards the biological material. The tip of the rolling head of the ball sticks to the microbiological colony which may then be either transferred or may be spread for microbiological cultures.

The diameter of the rolling ball 102 is 0.5 cm. It fits in the socket from the narrow side with 0.25 cm inside the socket and 0.25 cm outside the socket. The socket has two sides the narrow side (103) is the lower side with 0.6 cm width while the width of the upper side (104) is 0.75. The depth of the socket (101) is the distance between the upper and the lower side (210) and is equal to 1.0 cm. This depth allows each rolling head to fit in the other rolling head to sort them in a set. To stack the rolling balls on top of each other the top of the socket has an opening of 0.65 cm (205). This set can fit in pen holder hand. The dimension of pen holder hand is adequate with the size of the rolling head. It is 1.0 cm width (205) and 15 cm height (201). The holder hand can be providing in different shape. It is 15 cm long (302) with a head size 0.64 cm that fit in the rolling head (301).

The UV light source may be located near the open proximal end 205 of the pen holder device which may aid in sterilizing the ball of the rolling pen apparatus just before it is used. The UV-light source will re-sterilize/sterilize the rolling pen apparatus just before it is used for experimentation. Further, the pen chamber may also be sterilized before loading the rolling pen apparatus by using the UV light for a specific time and at a specific wavelength. The pen chamber may also be sterilized after the experiment is over to increase the shelf-life of the pen chamber and to maintain sterility of the pen chamber. The UV light source may also be located near the distal end of the pen chamber. Further, one may also place the UV light source at any other location on the outer wall, in between the outer and inner wall or the entire path of pen chamber. This will aid in maintaining highly aseptic environment while working with the microbiological colony. The UV light source may be of c-band width so that it may kill mold and mold spores as well as microbial contamination. A wire connects the ON and OFF switch button to the UV light source. The pen chamber may be made up of ethanol resistant material such as aluminum, synthetic composite and others.

The rolling pen apparatus may be developed as a single disposable unit or may be a number of disposable units sterilized and packed together. A number of rolling pen apparatus may be put together one over the other in the pen chamber. The assembly of multiple rolling pen apparatus is useful in carrying out work at a large scale or while working with a number of samples. It prevents cross-contamination as a user does not have to load single rolling pen apparatus each time and also because the UV light source re-sterilization is simultaneously occurring. The rolling pen apparatus via the pen holder device may be used to transfer microbiological colony such as bacteria from a first location to a second location along with even spreading of microbiological colony for a microbiological culture growth.

In some embodiments, the rolling pen apparatus can also be used via a holder hand device. A holder hand device as shown in FIG. 3 comprises of a holder 301 and a long extended hand 302. The holder 301 is cylindrical in shape and is used to attach or hold a rolling pen apparatus. The holder 301 holds or attaches a single rolling pen apparatus at one given time as shown in FIG. 4. The holder 301 is a hollow cylindrical tube with an outer wall and an inner wall. The hollow tube of the holder is for the housing of a single rolling pen apparatus. The diameter of the hollow cylindrical tube is more than the diameter of the outer body of the socket of the rolling pen apparatus. This allows for the easy attachment of the rolling pen apparatus into the holder hand device. Further, the holder may be of a shape depending on the shape and size of the rolling pen apparatus so that the holder can hold the rolling pen apparatus firmly for and during biological material transfer or other microbiological experiments.

A hand 302 of the holder hand device is a long extended structure. The hand 302 is of an appropriate length so that it can be held by a user's hand comfortably during experimentation. The hand 302 may also comprise of extensions to increase the length of the hand as per the user's need. As shown in FIG. 4, a rolling pen apparatus may be inserted into the holder 301 of the holder hand device. Further, the rolling pen apparatus may also be attached to the holder 301 of the holder hand device. The rolling pen apparatus may be inserted into the hollow cylindrical tube of the holder and thus the rolling pen apparatus and the holder hand device may be made as one single unit to use. The holder of the holder hand device may then hold the rolling pen apparatus during its use. The holder hand device and the rolling pen apparatus may also be sterilized as a single unit before use or may be sterilized as separate units and then combined as a single unit for use. The extended length and the shape of the hand make it easy and comfortable for a user to do the microbiological culture work without cross-contamination.

The holder hand may be made of plastic, metal, aluminum or other preferable material. The holder hand device may be packed as pre-sterilized units, re-usable units or disposable units. The holder hand device and the rolling pen apparatus may be sterilized and packed as a single unit and thus a user can open the pack just before use, use the single unit for experimentation and either discard it or re-sterilize it. The use of the holder hand device with a rolling pen apparatus helps working with microbiological colony easy convenient and provides good aseptic conditions. The long extended hand of the holder helps in keeping the hand of the user at a good distance while working with the microbiological colony.

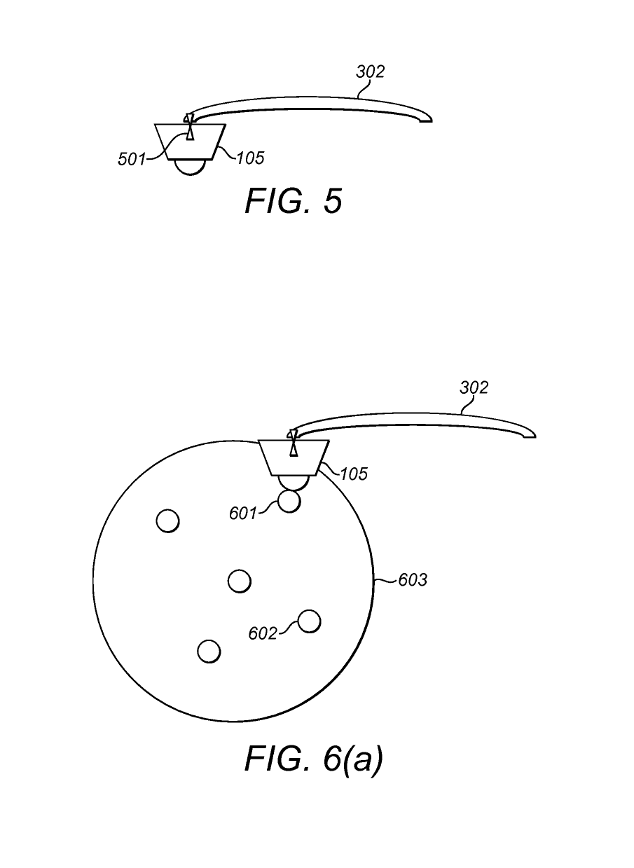

FIG. 5 is a side-view of a holder hand along with a rolling pen apparatus showing that the hand is attached to the holder via a hinge 501. The hinge 501 provides flexibility to the holder hand device and helps in the coordination of the holder and the hand while loading/unloading the rolling pen apparatus and coordination of holder hand device with rolling pen apparatus during other microbiological experiments. The hand 302 of the holder hand may be moved up, moved down and sideways during the microbiological experimentation with the flexibility provided by the hinge. The holder hand device may be a single unit with a hinge or may be two separate parts as a hand and a holder which may be attached together during the experimentation with the help of the hinge. The hinge 501 at any time has one part attached to the holder and one part attached to the hand.

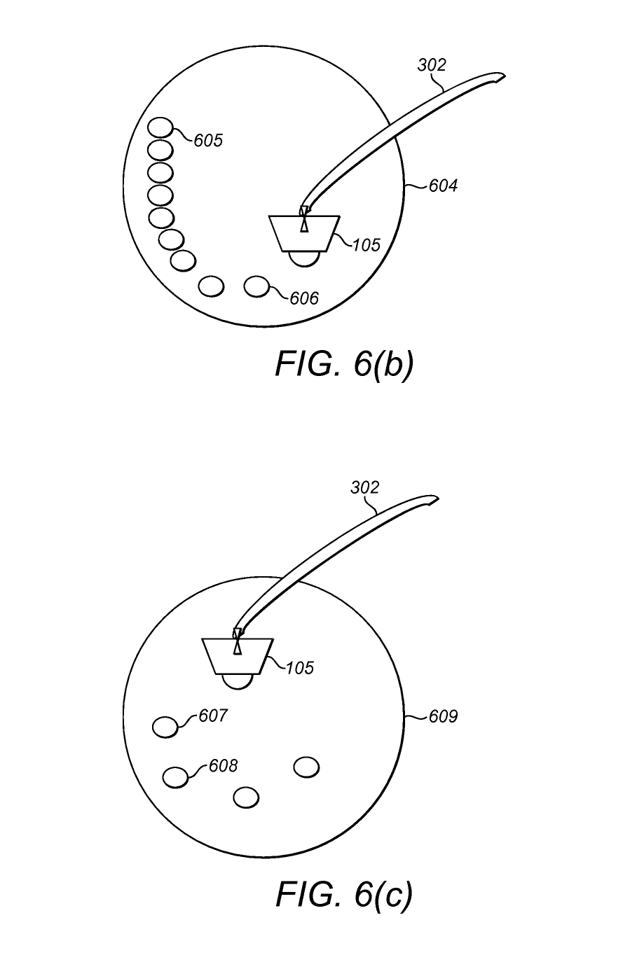

Some embodiments of the present invention further disclose a method of using the holder hand with a rolling pen apparatus attached to it. As shown in FIG. 6, the apparatus as disclosed in the present invention may be used to transfer microbiological colony such as a bacterial colony 601, 602 a first location to a second location. As shown in FIG. 6(a) the holder hand device with rolling pen apparatus is placed near and on a bacterial culture 601 grown on a solid media such as agar in a first petri plate 603 to pick up the bacterial colony via the tip of the rolling head of the rolling pen apparatus. The holder hand device is then moved onto a second petri plate 604 as shown in FIG. 6 (b) and the bacterial culture is spread 605, 606 onto the solid media such as agar in another petri plate 604. The rolling head of the rolling pen apparatus aid in evenly spreading of the microbiological colony as shown in FIG. 6(b) onto the solid media for growth. Also, the presence of the hinge in the holder hand provides flexibility to the holder hand making it easy to move the rolling pen apparatus down, up or in any desired direction during the microbiological experiments. Further, the holder hand device with the bacterial culture attached to the head of the rolling pen apparatus may also be moved to a another petri plate 609 as shown in FIG. 6 (c) and the holder hand device may then be placed as dots or points onto the solid media instead of streaking the culture. This aid in the development of the single bacterial colony instead of a loud of culture. This unique feature of rolling pen apparatus helps in the growth of a single bacterial colony which is very important for certain microbiological experiments.

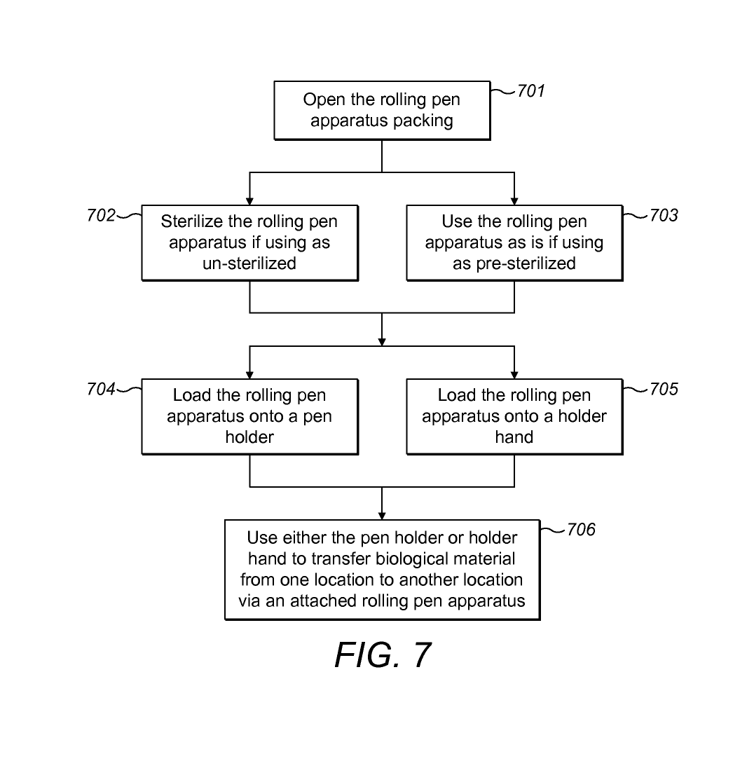

FIG. 7 shows a flow-chart diagram for using the disclosed rolling pen apparatus with a pen holder device or with a holder hand device. A user may open a rolling pen apparatus packing 701 which may be an un-sterilized packing 702 or a pre-sterilized packing 703. For the experimentation, the rolling pen apparatus may be load onto a pen holder device 704 or on a holder hand device 705 and may be used for its functioning i.e. to transfer biological material a first location to a second location 706. Also, the rolling pen apparatus may be further used to develop microbial cultures and a single microbial culture or a single bacterial colony.

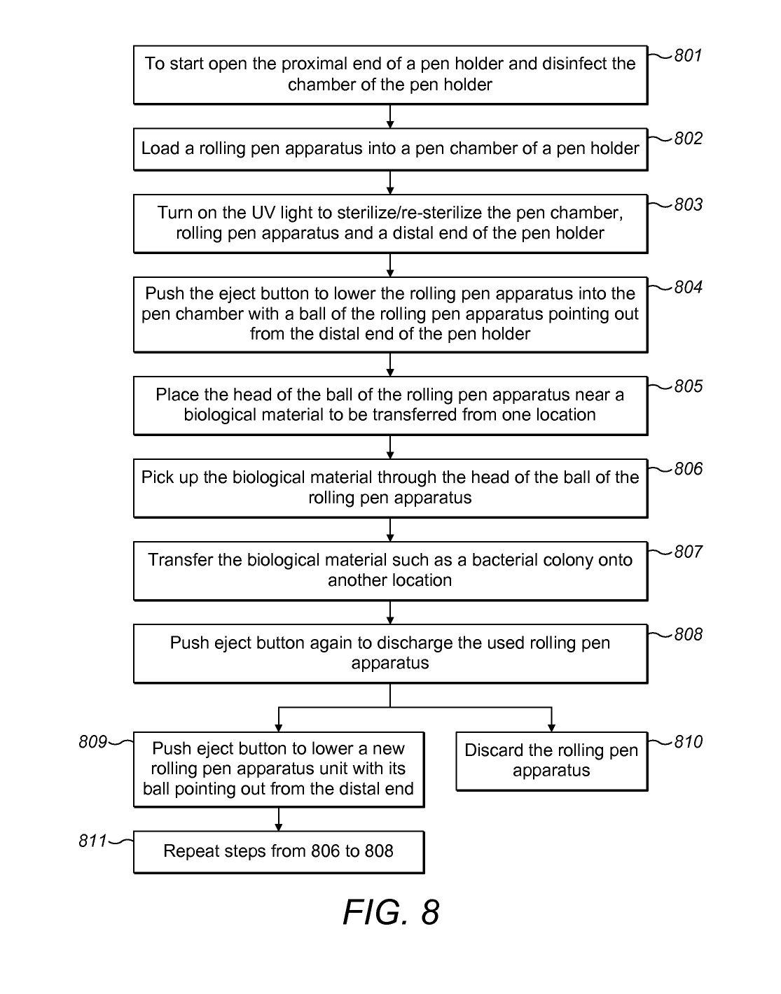

FIG. 8 shows a flow-chart for using the disclosed rolling pen apparatus with a pen holder device. To start working with the rolling pen apparatus with a pen holder device, a user may open a proximal end of the pen holder and disinfect the pen chamber 801 with an alcohol or another known disinfectant. Following disinfection, load the rolling pen apparatus into the pen chamber for its use 802. One may load a single apparatus unit into the pen chamber or a number of apparatus units depending on the experimental needs. Turn ON the UV light by an ON/OFF button located on an outer wall of the pen holder device 803. The UV light is turned ON to sterilize or re-sterilize the rolling pen apparatus, the pen chamber and the distal end of the pen holder device 803 before the start of the experimentation. Following the sterilization and re-sterilization process the UV light is turned OFF. Push the eject button to lower the rolling pen apparatus unit into the pen chamber with a ball of the rolling pen apparatus pointing out from the distal end of the pen holder device 804. Place a head of the ball of the rolling pen apparatus near and onto a biological material to be transferred 805 and pick up the biological material such as a microbiological colony through the head of the ball of the rolling pen apparatus from a first location 806. Transfer the biological or microbiological colony such as a bacterial colony onto a second location 807. Push the eject button again to discharge the used rolling pen apparatus 808. The user may discard the rolling pen apparatus 810 at the end of the experimentation or may push the eject button to lower next rolling pen apparatus unit for further experimentation 809 followed by repeating the steps as disclosed above 809.

Thus, a user may use a pen holder device with a rolling pen apparatus to transfer a biological material or a microbiological colony such as a bacterial culture a first location to a second location. A pen holder makes it very convenient for a user to work with the microbiological colony in a laminar flow unit or a hood and thus helps in working under aseptic conditions.

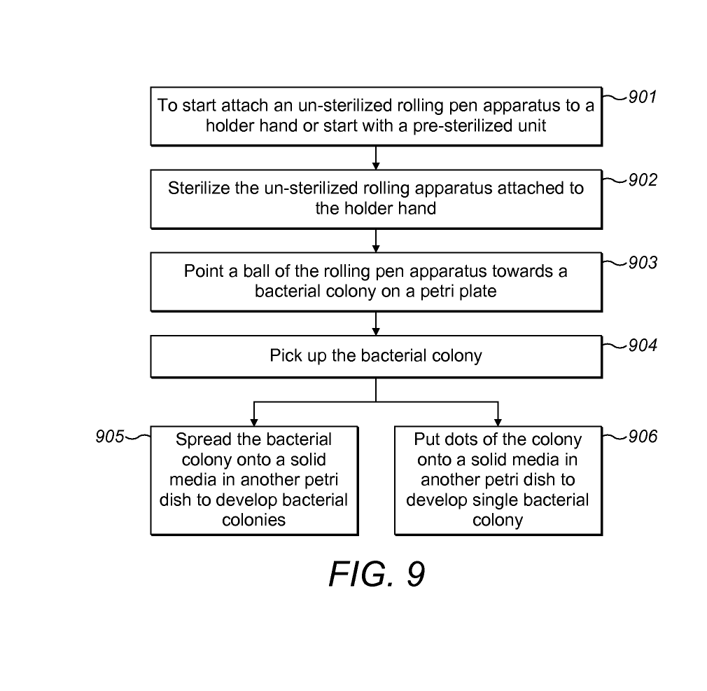

FIG. 9 shows a flow chart of using a holder hand device with a rolling pen apparatus for its use of preparing a single bacterial colony. To start the experimentation, a user may attach an un-sterilized or a pre-sterilized unit of the rolling pen apparatus with the holder hand device 901. If using an un-sterilized unit of the rolling pen apparatus, a user may sterilize it before the start of the experimentation or before getting in contact with a microbiological colony 902. Point a ball of the rolling pen apparatus towards a bacterial colony onto a solid media in a first petri plate 903. Pick up the bacterial colony with the ball of the rolling pen apparatus 904. Now, a user may spread the bacterial colony into a second petri plate onto a solid media such as an agar 905 or put dots/points of the material into a second petri plate onto the solid media for the growth of bacterial colonies or a single bacterial colony 906.

Although the present embodiments have been described with reference to specific example embodiments, it will be evident that various modifications and changes may be made to these embodiments without departing from the broader spirit and scope of the various embodiments. Accordingly, the specification and drawings are to be regarded in an illustrative rather than a restrictive sense.

### INDUSTRIAL APPLICABILITY

The presently disclosed apparatus finds a number of utility in microbiological laboratory procedures such as in developing single bacterial colonies, in transferring the biological material in solid or semi-solid form a first location to a second location with no cross-contaminations as it eliminates the steps of sterilizing the apparatus after every single use thus helps in maintaining aseptic conditions. This may also be used to propagate low cell volume from one place to another to make a monolayer spread of the cell for propagation.

The rolling head of the rolling pen apparatus helps in evenly spreading/streaking the microbiological colony onto the solid media such as agar for the culture growth and is a great advantage in microbiological work. The disclosed apparatus also helps in maintaining health safety of users working in microbiology laboratories or other settings where they come in contact with hazardous biological material. Further, the apparatus and methodology disclosed in the present application also finds utility in biotechnology procedures and experiments.

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A rolling pen apparatus, comprising:
a socket; and
a ball inside the socket wherein the rolling pen apparatus is used to transfer a biological material from a first location to a second location.

22. The rolling pen apparatus of claim 21, wherein the socket comprises:
a proximal end; and
a distal end wherein the proximal end has a ball inserted into the proximal end to evenly spread a biological material onto a solid media surface.

23. The rolling pen apparatus of claim 21, wherein the ball comprise of a rolling head to enable the ball to move in a forward direction or a backward direction.

23. The rolling pen apparatus of claim 21, wherein the ball comprise of rolling head to move the ball in a desired direction.

24. The rolling pen apparatus of claim 21, wherein the said rolling pen apparatus is used to develop a single microbiological colony.

25. The rolling pen apparatus of claim 21, wherein the first location is a location where biological material is present.

26. The rolling pen apparatus of claim 21, wherein the second location is the location where biological material is to be transferred.

27. The rolling pen apparatus of claim 21, wherein the biological material is a microbiological material.

28. A holder hand device, comprising:
a holder; and
a long extended hand wherein the holder hand device holds the rolling pen apparatus within the holder for its use.

29. The device of claim 28, wherein the holder and long extended hand is held together via a hinge.

30. The device of claim 28, wherein the holder hand device holds the rolling pen apparatus within the holder to transfer microbiological material from a first location to a second location.

31. The device of claim 28, wherein the holder hand device holds the rolling pen apparatus within the holder to develop a microbiological culture.

32. The device of claim 28, wherein the holder hand device holds the rolling pen apparatus within the holder to develop a single bacterial colony.

33. A method, comprising:
(a) opening a proximal end of a pen holder device;
(b) disinfecting a pen chamber;
(c) loading the rolling pen apparatus into the pen chamber;
(d) turning ON a UV light to sterilize the rolling pen apparatus, the pen chamber and the distal end of the pen holder device;
(e) pushing an eject button to lower the rolling pen apparatus into the pen chamber;
(f) placing the rolling pen apparatus near a biological material to be transferred from first location;
(g) picking up the biological material;
(h) transferring the biological material onto second location; and
(i) pushing the eject button again to discharge the used rolling pen apparatus wherein the pen holder device aid in moving the rolling pen apparatus for its functioning.

34. The method of claim 33, wherein the first location is where the biological material is present.

35. The method of claim 33, wherein the second location is where the biological material is to be transferred.

36. The method of using the rolling pen apparatus of claim 33, wherein the pen holder device aid in preparing a single bacterial colony by moving the rolling pen apparatus.
